(19) United States
(12) Patent Application Publication
(10) Pub. No.: US 2024/0347185 A1
(43) Pub. Date: Oct. 17, 2024

(54) SURGICAL SUPPLY AND REPROCESSING SYSTEM

(71) Applicant: Bedrock Surgical, Inc, Palo Alto, CA (US)

(72) Inventor: Shawn Flynn, Portland, OR (US)

(73) Assignee: Bedrock Surgical, Inc, Palo Alto, CA (US)

(21) Appl. No.: 18/682,912

(22) PCT Filed: Aug. 11, 2022
(86) PCT No.: PCT/US22/40136

§ 371 Date: Feb. 10, 2024

Related U.S. Application Data
(60) Provisional application No. 63/232,093, filed on Aug. 11, 2021.

(51) Int. Cl. G16H 40/20; A61B 34/10

(52) U.S. Cl. CPC G16H 40/20; A61B 34/10

(57) ABSTRACT

Through the data collected at multiple points of the SSR system, it will be possible to make an optimized set of instruments available in a timely manner while ensuring that each instrument is at or above a measurable quality criterion. The SSR can include a networking capable computer, computation engine, storage and input output facilities incorporating an array of sensors. The SSR can support multiple “stations” each of which can gather data or provide information at the various steps within the workflow.

### CROSS-REFERENCES

This application claims the benefit of U.S. Provisional Application No. 63/232,093, filed Aug. 11, 2021.

### BRIEF DESCRIPTION OF THE DRAWINGS

Various example embodiments can be more completely understood in consideration of the following detailed description in connection with the accompanying drawings, in which:

FIG. 1 shows a diagram of a typical instrument reprocessing workflow.

FIG. 2 shows example of a surgical supply and reprocessing system implementation.

FIG. 3A shows further details for an example of a surgical supply and reprocessing system implementation.

FIG. 3B is a flow diagram that shows another example of a surgical supply and reprocessing system implementation.

FIG. 4 is a flow diagram that shows how instruments can be managed based on probability of use and personalization.

FIG. 5A shows an example where relevant information about an instrument is available at an appropriate station.

FIG. 5B shows an example where relevant information about an instrument is available at an appropriate station in greater detail.

FIG. 5C shows an exemplary implementation of a context sensitive instruction system for surgical supply reprocessing.

FIG. 6A is a flow diagram showing exemplary steps of onboarding of instruments and an interactive inventory database.

FIG. 6A is a flow diagram showing exemplary steps of post onboarding of instruments.

FIG. 7 shows one embodiment of a surgical supply and reprocessing system (SSR) system and its architecture.

FIG. 8A shows one possible configuration of SSR with the use of off premise reprocessing and storage.

FIG. 8B shows another possible configuration of SSR from FIG. 8A but with further possible optimizations for reprocessing workflow.

### DETAILED DESCRIPTION

This disclosure describes inventive concepts with reference to specific examples. However, the intent is to cover all modifications, equivalents, and alternatives of the inventive concepts that are consistent with this disclosure. It will be apparent, however, to one of ordinary skill in the art that the present approach can be practiced without these specific details. Thus, the specific details set forth are merely exemplary, and is not intended to limit what is presently disclosed. The features implemented in one embodiment can be implemented in another embodiment where logically possible. The specific details can be varied from and still be contemplated to be within the spirit and scope of what is being disclosed.

Instruments that are used in medical procedures such as surgery, need to be cleaned and sterilized prior to use. Hospitals and other facilities such as outpatient surgery centers, typically have dedicated resources including staff and cleaning and sterilization equipment ensuring that the instruments are cleaned, disinfected and are in proper working condition. While there is centralization and standardization of resources to handle the instruments, at the operating room or other locations where the instruments are used, each doctor, each patient and each procedure imposes a different set of requirements that does not support centralization and standardization.

The system called the “Surgical Supply and Reprocessing system” or SSR provides the architecture for an instrument reprocessing workflow. Instruments that are used in medical procedures such as surgery, need to be cleaned and sterilized prior to use. Hospitals and other facilities such as out-patient surgery centers, typically have dedicated resources including staff and cleaning and sterilization equipment ensuring that the instruments are cleaned, disinfected and are in proper working condition. While there is centralization and standardization of resources to handle the instruments, at the operating room or other locations where the instruments are used, each doctor, each patient and each procedure imposes a different set of requirements that does not support centralization and standardization. Most hospitals lack sophisticated inventory tracking systems for surgical instruments. They also lack system for inspecting instruments and instrument sets for contamination or impending failure.

In order to attend to the patients' needs, the sterile processing department (SPD) associated with the hospitals and clinics oversupply the doctor and his or her medical staff with instruments at the operating room. This practice has led to significant inefficiencies, straining the resources of the SPD in terms of stress on the staff, equipment and the economics of running the SPD. As an example, according to Mhalaba et al. (Mhalaba et al., “Surgical instrumentation: the true cost of instrument trays and a potential strategy for optimization”, Journal of Hospital Administration, 2015, 4 [6]: 82-88.), only 29% of instruments sent to the operating room for a major laparotomy procedure are actually used.

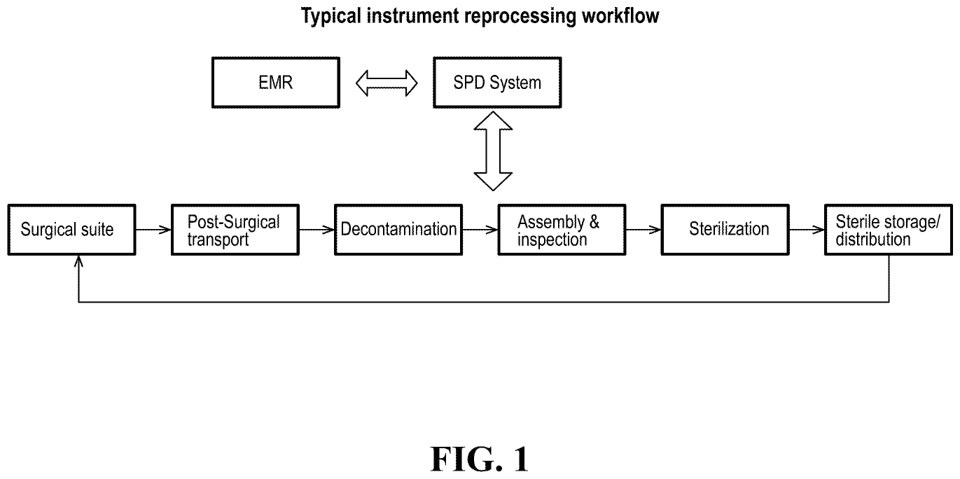

Through the data collected at multiple points of the SSR system, it will be possible to make an optimized set of instruments available in a timely manner while ensuring that each instrument is at or above a measurable quality criterion. The SSR can include a networking capable computer, computation engine, storage and input output facilities incorporating an array of sensors. The SSR can support multiple “stations” each of which can gather data or provide information at the various steps within the workflow. The sensors can gather information about specific activities at each station. Different sensors can be used at each station. The sensors can be of various types including but not limited to optical sensors using various wavelengths for detection of a variety of parameters, chemical sensors or mechanical sensors. The instruction and information station can include a computer with typical accessories such as a display, keyboard and a mouse, can also be deployed at each station and can provide appropriate information at the appropriate time. The onboarding system can be deployed at the assembly and inspection station and can provide a method to onboard or register each instrument in a visual manner as will be described below. Finally, the cloud system provides a way to perform analysis and gather data from other facilities using the SSR. FIG. 1 outlines a typical instrument reprocessing workflow within and outside SPD. In general, Electronic Medical Record (EMR) system allows the medical staff to schedule and plan for procedures. As part of this planning process, a doctor is able to specify instruments they prefer using. The preference is collected in a preference card. These cards can be maintained as physical cards or electronic cards kept within a computer system. Typically, these cards are not well maintained and do not get updated as the needs of the doctor and the patients change. The preference card and the surgery schedule are some factors that drive the workflow within the SPD. Within the workflow, instruments are decontaminated in the decontamination station. The decontamination station typically involves hand washing and/or scrubbing of instruments, bathing the instruments in a disinfecting agent, and pressure washing in automatic instrument washers. Each instrument should be processed according to instructions provided by the manufacturers in an instruction manual called Instructions For Use (IFU). However, in today's environment, the work product of reprocessing workflow depends to a very large extent on the expertise and the knowledge of the technicians handling the instruments at each stage of the workflow. IFUS are long and detailed. Pressure for rapid processing of large sets of instruments does not provide time for operators or technicians to read and cross reference procedures with IFU instructions. IFUs are not reviewed regularly and changes to the IFU can go unnoticed There is no systematic vehicle for cross checking procedures with IFU prescribed actions. There is also no standardized procedures for validation and verification if the procedures occurred as prescribed. Following decontamination, the instruments are transferred to an assembly and inspection station. Here the washed instruments are inspected for damage and if they have been cleaned adequately. The instruments are then assembled in groups or sets according to what can be needed in a procedure in the operating room (OR). Sets are determined by generalized preferences of a group of surgeons in order to standardize the sets. These generic sets increase the number of instruments required and increase the ratio of instruments processed relative to instruments used. In addition, in today's environment, after the washing cycle, typically visual inspections are conducted aided by microscopes. The assembly of sets also is done manually. The assembly is done according to predetermined lists that would have been created by analyzing the historical usage pattern for a particular procedure and with the preference card. However, these lists are not dynamic and there are no convenient ways to modify them. As one of the final steps in the assembly and inspection station, the set of instruments is packaged in a “peel pack” or other suitable packaging containers. Subsequently, the packaged instruments are sent to a sterilization station. Several methods of sterilization can be used according to the specifications in the IFU including steam-based methods (high temperature) and plasma (low temperature) based methods. Following sterilization, the set of instruments is sent to storage or sent in “case carts” to the surgical suite or the operating room. Case carts contain all the sets of instruments and the soft goods required for a particular procedure, soft goods include gowns, gloves, masks, etc.

At the surgical suite, a circulating nurse hands the instruments from the case cart to the scrub nurse who lays the instruments out on a back table within a sterile field. A secondary cart, frequently called a Mayo stand can also be used by the bedside (also within the sterile field) to transfer instruments from the back table to the Mayo stand. Many issues are experienced at the surgical suite. For example, the peel pack can be torn in the process of transportation to the surgical suite, negating the sterilization status of the instruments. The sets may not have the right list of instruments. The instruments can still contain biomass from a previous procedure. The surgical team has to make a determination based on the status of the patient and the instruments whether to proceed with the procedure.

From a safety and quality standpoint, the opening of the trays in the surgical suite is a critical expensive and unreliable stage in the tool preparation process. The scrub nurse is performing the function of opening and inspecting each tray to confirm that the tray has not been damaged and to confirm that the indicators suggest that the sterilization process was performed properly. There are extreme time pressures on staff in the OR and the packaging and inspection must be done quickly which undermines the thoroughness of the inspection, the lack of attention to sterile technique increases opportunities for contamination or dropped instruments.

After the surgical procedure has been completed, the instruments are placed in containers and are transported back to the decontamination station. Regulations imposed by agencies and procedures adopted by hospitals, generally require that the instruments are reassembled back in sets and are pre-treated in a disinfectant agent before they are sent back to decontamination. However, in this step as in other places within the workflow, the efficiency and adherence to procedures depends on the skill and training of the personnel.

On an instrument level, in today's environment, instruments often are lost or misplaced due to the sheer volume of the operation. Instruments are incorrectly placed in a different set. This can happen post-surgery or at assembly. They are sometimes lost within the automatic washer. If an instrument is lost, the SPD loses valuable time for getting the instrument to the surgical team in time. These sorts of issues place enormous burden on the system in terms of cost and efficiency.

Most hospitals lack sophisticated inventory tracking systems for surgical instruments. They also lack system for inspecting instruments and instrument sets for contamination or impending failure.

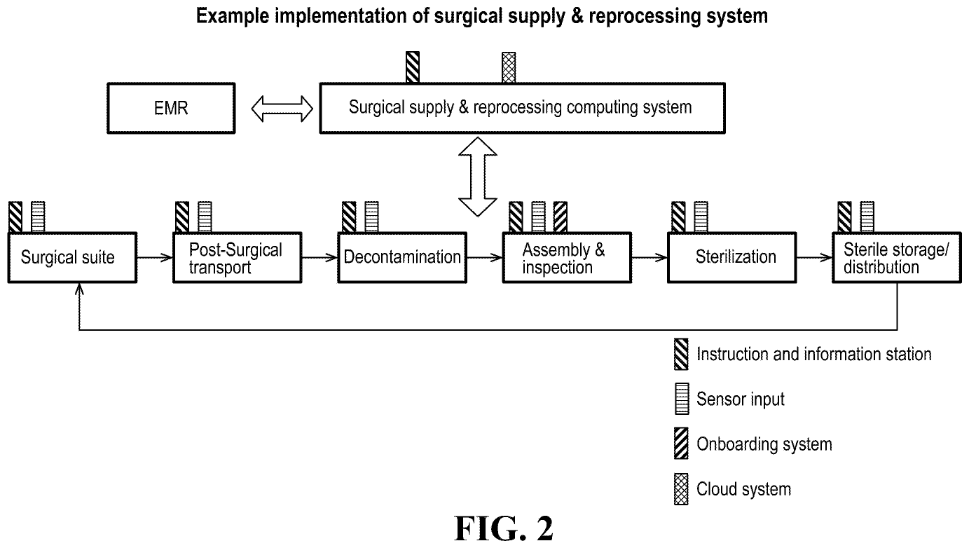

FIG. 2 illustrates an improved system called “the surgical supply and reprocessing system” or SSR that overcomes the shortcomings described above. The SSR can include a networking capable computer, computation engine, storage and input output facilities incorporating an array of sensors. The SSR can support multiple “stations” each of which can gather data or provide information at the various steps within the workflow. The sensors can gather information about specific activities at each station. Different sensors can be used at each station. The sensors can be of various types including but not limited to optical sensors using various wavelengths for detection of a variety of parameters, chemical sensors or mechanical sensors. The instruction and information station can include a computer with typical accessories such as a display, keyboard and a mouse, can also be deployed at each station and can provide appropriate information at the appropriate time. The onboarding system can be deployed at the assembly and inspection station and can provide a method to onboard or register each instrument in a visual manner as will be described below. Finally, the cloud system provides a way to perform analysis and gather data from other facilities using the SSR.

### Dynamic Packaging

A system and technique to reduce the cost of reprocessing without giving up the flexibility of having an instrument at hand when needed is now described. There have been a number of studies that demonstrate that only a small percent of instruments included in a tray or set is used in any specific procedure. For example, Mhalaba et al. state that out of 10 major laparotomy cases observed, only 29% of the trays were used with a standard deviation of +/−10% and a range of 9%-43%. This provides the concept to reduce the instruments in a set however there is a risk of not including an instrument in a tray even if it is rarely used—the patient's life could be at stake. Thus, the concept below ensures that all instruments as per current practice, are available when needed however the cost and inefficiency of processing these instruments is minimized.

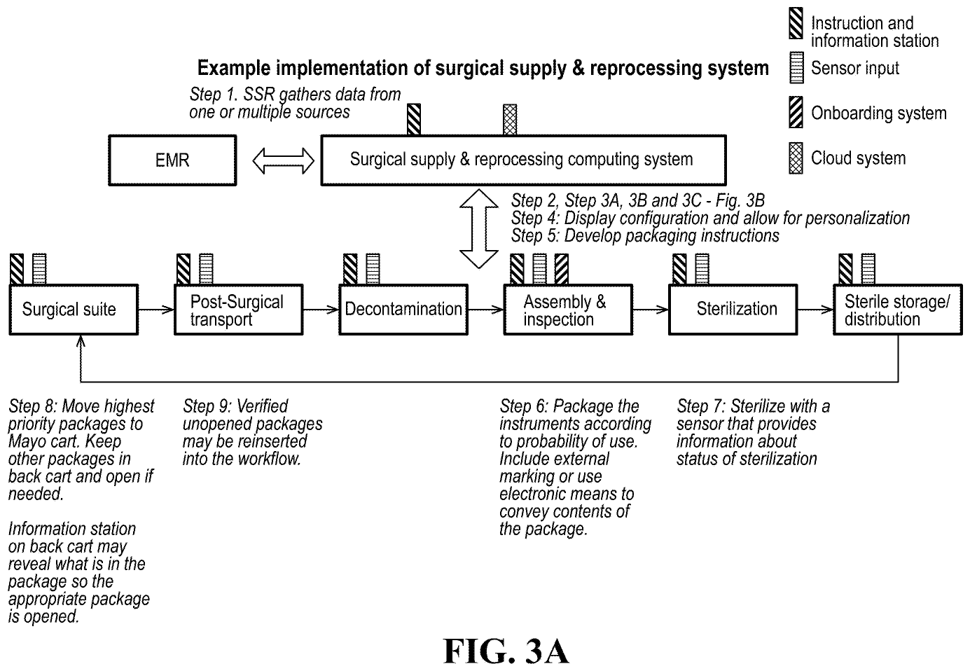

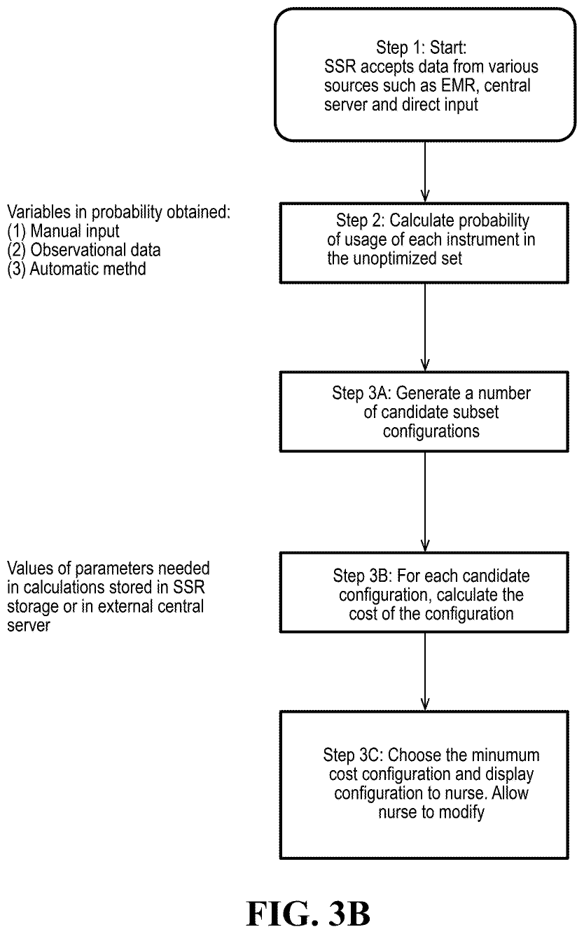

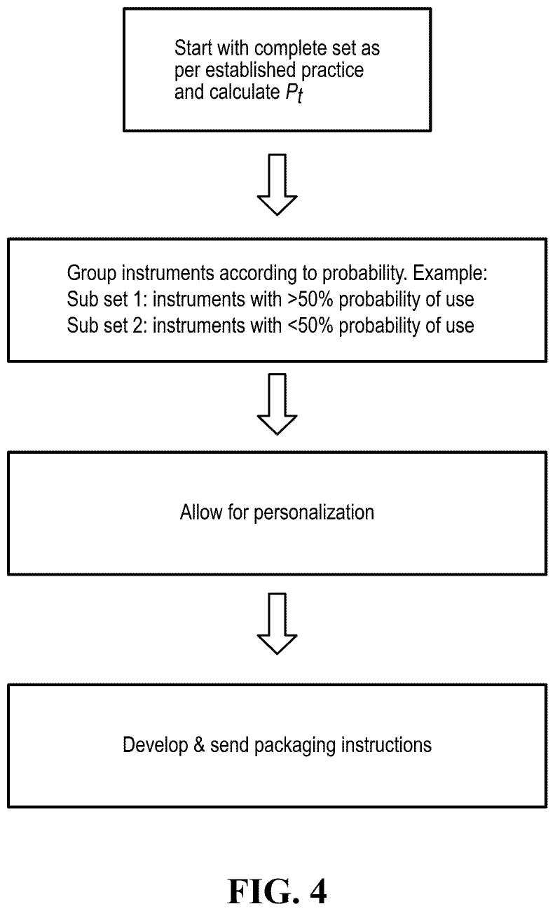

FIGS. 3A and 3B illustrate this concept and an example implementation. FIG. 3A describes the overall concept and how it is implemented with the SSR and FIG. 3B provides more detail on one aspect of the implementation. Referring to FIG. 3A, in brief, the probability of usage of each instrument in a tray configured as per the current procedure, is calculated. Subsequently, the instruments are divided into one or multiple subsets where each subset includes instruments in specific range of probabilities of use. The SSR system calculates the cost of various configurations of the subsets. The configuration with minimized or reduced cost is then packaged and sterilized and sent to the surgical suite. It may be that the instruments with the smaller probability of use may not be opened at the surgical suite but is close at hand if needed however the sterilization remains intact. The unopened subsets can be returned to the SPD but reinjected in the workflow without re-sterilization or any other handling within the SPD (other than transportation). This can lead to reducing the cost of operations and retains the ability of the surgical team to access an instrument if needed. The details of an example implementation are now described. Variations of this implementation are also possible.

The following steps are in reference to FIG. 3A and FIG. 3B.

### Step 1 (Data Gathering Step):

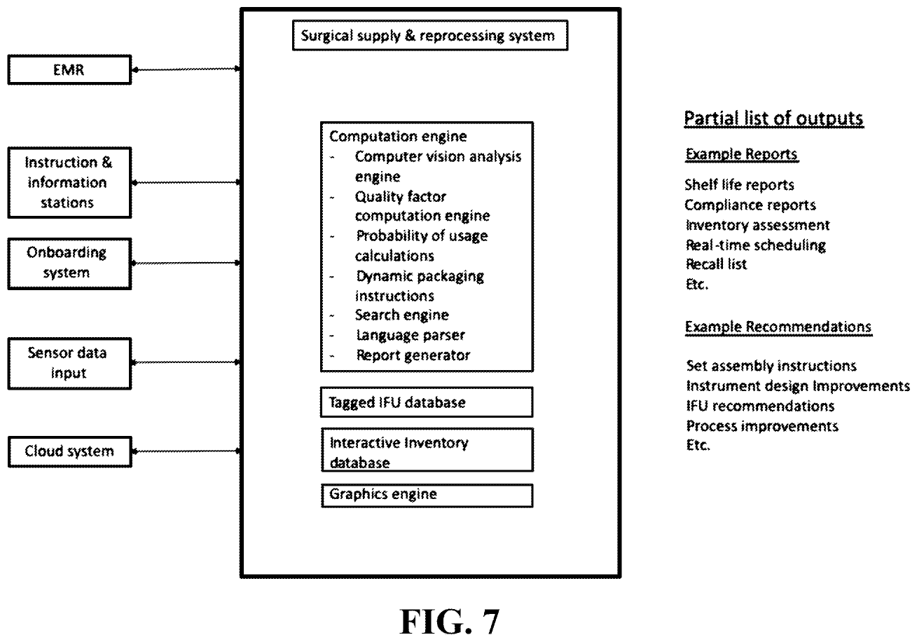

In this step, the SSR system accepts data from one or multiple sources. Data about the procedure, about the patient, the surgical team that is performing the procedure, can be gathered from the EMR or other scheduling system or database. Data can also be stored in an external central server that the SSR system can access. The external central server can act as a central storage and computing facility to multiple SSR systems across different facilities. The architecture of the SSR system including the central server is described later in FIG. 7. Data such as data from the surgical team, can also be directly input at the SSR through one of the instructions and information stations.

### Step 2 (Calculate the Probability of Usage for Each Instrument):

This step can be performed by the computing engine within the SSR. In this step, a set of instruments is requisitioned as per current operating procedure. For the purposes of this discussion, this initial set will be referred to as the “unoptimized set”. Typically following current practice, the unoptimized set can contain multiple and potentially large number of instruments, many of which may not be used in the surgical suite. Next, for each instrument in the unoptimized set, the probability of use is calculated by the SSR.

### Calculating the Probability of Usage

The probability of usage Pᵢ for any particular instrument i can be dependent on various factors such as the physician who is using the instrument, the surgery being performed, the hospital where the procedure is occurring etc. One technique to include these variations, is to calculate or estimate the joint probability P_{(l,j,k,l . . . )} for any particular instrument for the use case j,k,l etc. The underlying statistics can be found by recording usage data for every instrument, while simultaneously recording additional details associated with that use instance. In this way, the instrument can be associated with a particular individual, hospital, or procedure. The underlying statistics, once obtained, allows for the computation of any two events happening simultaneously (doctor & procedure, procedure & hospital, instrument & doctor, etc):

Thus:

$${P}_{( i , x )} = \frac{\# of times instrument i is used when x = \{ j , k , l … \}}{\# of times instrument i is used included in a set} \qquad Eqn . 1$$

This equation can be used to accommodate various factors such as the criticality of the instrument to a particular procedure or to a portion of the procedure, how differentiated the tool is etc.

There are a number of methods to record the statistics used in Eqn. 1. Three methods are described below. In the methods that that require a manual input or interpretation from a human expert, the SSR system would have a suitable input interface implemented on one of the instruction and information stations associated with the system to obtain the input.
a) Manual input: Once the unoptimized set has been requisitioned, a qualified personnel such as a scrub nurse familiar with the practices of the surgeon scheduled to perform the surgery, can enter a number of prior uses. An instruction and information station associated with the SSR system can display the list of instruments and require the scrub nurse to enter the probability manually.
b) Observational data: The probability can be calculated based on local observational data or data from multiple sites. With respect to the local observational data, the denominator in Eqn. 1 can be known within the scope of the current practice. To obtain the numerator in Eqn. 1, a nurse or technician can manually note the usage of each instrument either at the end of the surgery or when the instruments come back to decontamination. The SSR can group the observational data by surgical procedure, patient habitus, age group, or in various other ways. As observational data grows, these probabilities can become more robust. Robustness can also be achieved as the data from each SSR system is collected and analyzed by the external central server. The external central server can also group the data in various ways as noted above
c) Automatic method: In the case each instrument is coupled to a unique device identifier (UDI) and in particular if the entire workflow is sufficiently sensorized with the ability to detect and track each instrument, then the variables of Eqn. 1 can be obtained automatically. The sensorization and coupling of UDIs are described later.

### Step 3A (Generate Candidate Configurations):

In a subsequent step, an algorithm called the “optimization algorithm”, determines the configuration of an optimized set. There are various ways of optimizing how to partition the unoptimized set with one example described now. FIG. 3B illustrates this example algorithm. As described above, an initial step is to commence with the unoptimized set and calculate the probability as in Eqn. 1. Next, a number of candidate subset configurations can be generated. These subsets can be used to determine placement of groups of instruments in the OR. As an example, an optimized set can include two subsets, where one subset is placed on the Mayo stand by the bedside and is automatically opened for usage while the second set is placed in a temporary storage area and not automatically opened for each case but held in backup until requested. The temporary storage area can be the back table within the sterile field or in a storage facility away from the OR but still accessible in some definite and small period of time. This presorting of instruments into subgroups for specific placement in the OR can result in reduced OR setup time and can optimize the surgical workflows for reduced cost and improved operational workflow efficiency. To generate the candidate configurations, two variables can be considered (a) the number of subsets to partition the unoptimized subset into and (b) the probability levels for each subset defining which instruments can be included within a specific subset. A table such as Table 1 can be generated. As illustrated in this table, candidate configurations 1 and 2 each specify two subsets. Candidate 1 groups instruments with >0.5 probability of use into subset 1 and the rest in subset 2. Candidate 2 groups instruments with >0.75 probability of use into subset 1 and the rest in subset 2. Other probability threshold with two subsets can be explored. Candidate 3 groups the unoptimized subset into 3 subsets, each subset with the probabilities as illustrated. Candidates with more subsets and other thresholds can be explored.

TABLE 1

| | | Probability | Probability | Probability | |
|---|---|---|---|---|---|
| Candidate | # of | levels of | levels of | levels of | |
| configuration | subsets | subset 1 | subset 2 | subset 3 | . . . |
| 1 | 2 | >0.5 | <=0.5 | | |
| 2 | 2 | >0.75 | <=0.25 | | |
| . . . | | | | | |
| 3 | 3 | >.66 | <=0.66 | <=0.33 | |
| | | | & >0.33 | | |
| 4 | 3 | >0.5 | <=0.5 | <=0.25 | |
| | | | & >0.25 | | |
| . . . | | | | | |

### Step 3B (Cost Calculation) for Each Candidate Configuration, a Cost can Now be Calculated:

There are various methods to calculate the cost function. In addition, the more variables that influence the cost that are included in the cost calculations, the more representative the cost result will likely be. One example method for quantifying the cost-value of a candidate group is described below. Thus,

$${C}_{g} = {\sum }_{s = 1}^{S} {\sum }_{n = 1}^{{N}_{s}} {α}_{n , s} {‖ {C}_{n , s} ‖}^{2} , \qquad Eqn . 2$$

where C_{g} is the cost for a specific candidate configuration, and where the variable s represents the subset number, S is the total number of subsets, n represents the instrument number and N_{g} represents the total number of instruments in each group s. C_{g} is computed as a sum over optimization parameters C_{n,s} and weights α_{n,s}. These parameters can be used to customize the workflow for a particular surgeon, hospital, or procedure. The weights α_{n,s} in this equation provides a method to accommodate the differences in costs for a particular reprocessing workflow between each institution.

The set of cost parameters {C_{n,s}} are used to quantify the cost of each workflow step. Eqn. 2 provides the flexibility of not just capturing the cost of each step in the reprocessing workflow, but it also allows capturing the indirect costs such as cost for waiting for an instrument, cost of operating the surgical suite etc. Thus, these various factors can be conceptually through of a reprocessing workflow step. This equation can also be used to coerce the solution towards a more desirable configuration where the desirability can be defined in various ways including a configuration with the lowest reprocessing cost and the fewest instruments likely to require reprocessing. Other optimizations can also be possible such as having the fastest throughput the surgical suite.

The probability-weighted cost parameter for the n^{th} workflow step Cₙ for an instrument i in a subset s can be written as:

$${C}_{n , s} = {\sum }_{i = 0}^{M} {β}_{i , {p}_{i}} , {C}_{i} , \qquad Eqn . 3$$

is computed by multiplying the identity vector for the instrument Cᵢ and probability of use p_{i,x} (found in step 2 described above for the particular use case) with a pre-computed matrix β_{i,p}_{ᵢ} that maps the identity vector to a relative cost of processing each instrument placed in a subset s in that reprocessing step. The pre-computed matrix β_{i,p}_{ᵢ} can be associated in the instrument i and the probability of use for that instrument placed in a subset s. This type of formulation in Eqn. 3 allows the flexibility to accommodate for the effect of the probability on various reprocessing steps including the steps that are indirectly associated with the instrument reprocessing. As an example, β_{i,p}_{ᵢ} can be computed as pᵢβᵢ for a washing step however the cost of waiting for all instruments within a subset that is placed in a location a few minutes away from the bedside, can all be captured as a constant value (i.e. same for all instruments within that subset).

In review, these steps provide a technique to obtain a desired effect such as wanted to reduce or minimize the cost of reprocessing, by modelling various configurations of the subsets and estimating the cost of the configurations. The steps also provide a technique to accommodate the variations in the cost between institutions. Additionally, because any indirect costs or external optimization parameter (external to the steps performed within the reprocessing workflow) can be modeled as a workflow step. These include criticality of the surgical procedure, cost of operating the surgical suite, waiting time for the instruments etc.

### Step 3C (Choosing the Minimal Cost Option and Display):

After cost of the configurations are calculated, the minimal cost option can be chosen. Thus, steps result in a configuration of the unoptimized set in one or multiple subsets. These steps also allow the impact of keeping subsets 2 or greater at different locations so that when needed, they would be available as needed. Thus, in one example, if candidate 1 were chosen to be implemented, subset 1 can be kept on the Mayo stand (by the bedside and ready to be used) and subset 2 can be kept on the back table. The instruments in the back table in subset 2 would be available almost immediately when needed. However, the technique above allows exploration of scenarios where subset 2 and above were kept in a storage area that is not in the immediate vicinity of the surgical team.

### Step 4: Personalization:

After a candidate configuration has been selected, the SSR can display the packaging to a qualified person such as a scrub nurse familiar with a specific doctor's practice or preference. The nurse can alter the configuration manually based on various factors such as the doctor's preference. The SSR accepts this input and continues to the next step.

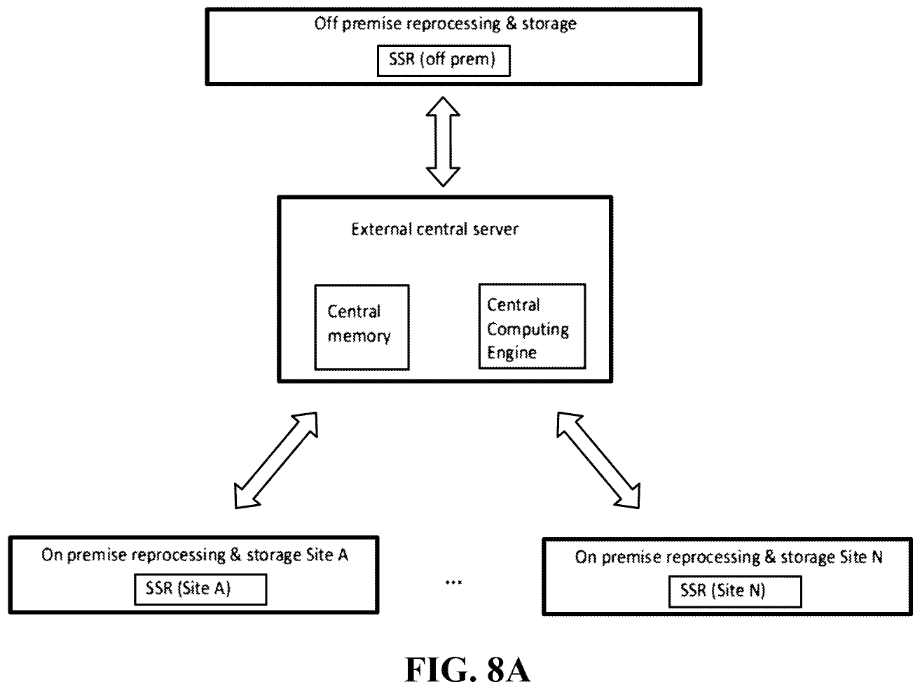

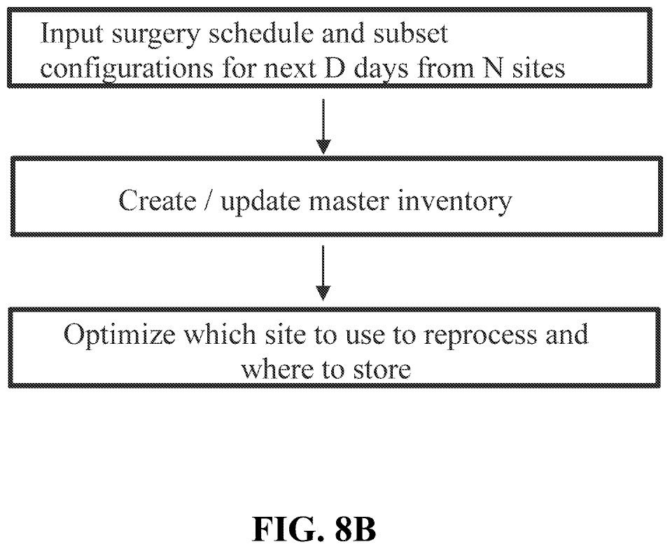

In addition to the personalization on a per surgeon level, is it to be noted that the personalization level can be done at more than an individual level with steps discussed so above. As an example, the use of joint probabilities in Eqn. 1, allows the personalization for each hospital or for each procedure or for a number of other variables that can be deemed relevant by the staff. The networking architecture of the SSR as will be discussed in relation to FIG. 8, allows the SSR system to also suggest factors that were seen to be relevant in other institutions using their version of the SSR system.

### Step 5: Developing Packaging Instructions:

As the subsets can differ from patient to patient even for the same type of procedure, specific packaging instructions can be generated by the SSR computing engine and sent to the assembly and inspection system. The packaging instructions can contain a list of instruments along with an image of each instrument that is to be included in each subset; this image and list can be sent to the operator at the assembly and inspection station.

### Step 6: Inspection and Packaging;

Here the operator inspects and packages the instruments as they come out from the washing station. Labels or other identifying marking can be placed on the package to alert or inform the nurses and staff at the surgical suite regarding the contents of each package. This marking can be in the form of a physical list or it can be an electronic list which can be displayed on the appropriate instruction and information station.

For the subsets that were returned from the surgical suite unopened, they can be injected into the workflow at the assembly and inspection station for a reverification of the status of sterilization. The verification can be done manually or with sensors. These instruments can be handled separately (within their sterilization package) and not mixed with the freshly washed instruments in the eventuality that the sterilization is negated. Once the sterilization status is verified, then the subsets can be mixed in with other subsets consisting of washed instruments however the unopened subsets can be sent to a case cart or to storage because they would not need sterilization.

One type of sensor and packaging that can be used to check for continued integrity of sterilization is a clear vacuum sustaining packaging with a vacuum sensor clearly visible from outside. The vacuum package can be applied over the traditional peel pack or other packaging and the sensor would be placed inside the vacuum package. This packaging would be applied at the sterilization station as part of the sterilization process.

### Step 7: Sterilization:

Sterilization can occur as per established practice.

### Step 8: Handling at the Surgical Suite:

Once the packages are sent to the surgical suite, it is advantageous for the staff to quickly know the contents and sterilization status of each package by reading the values of sensors that can be included in the packages. To this end, an instruction and information station can be installed on the back table that can display the contents and the sterilization status of each package. Thus, in the example of configuration 1 in Table 1, subset 1 can be transferred to the Mayo stand and subset 2 can be kept in the back table and only opened if needed.

### Step 9: Post-Surgery Transport:

Post-surgery, nurses or technicians can verify that the status of sterilization of the unopened subsets is not negated. After verification, these subsets can be sent to the SPD such that they are kept separate from the instruments that were used. These can be reinjected into the workflow at the assembly and inspection station as described above.

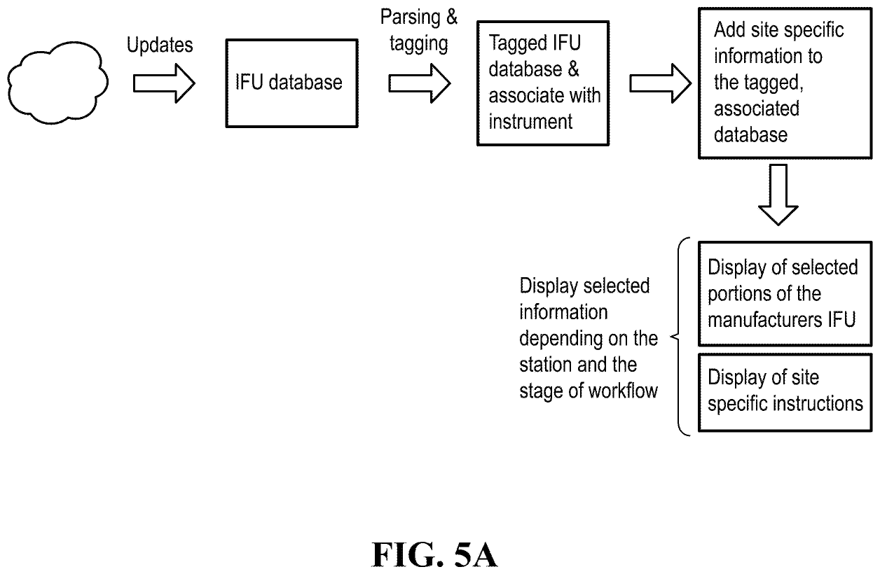

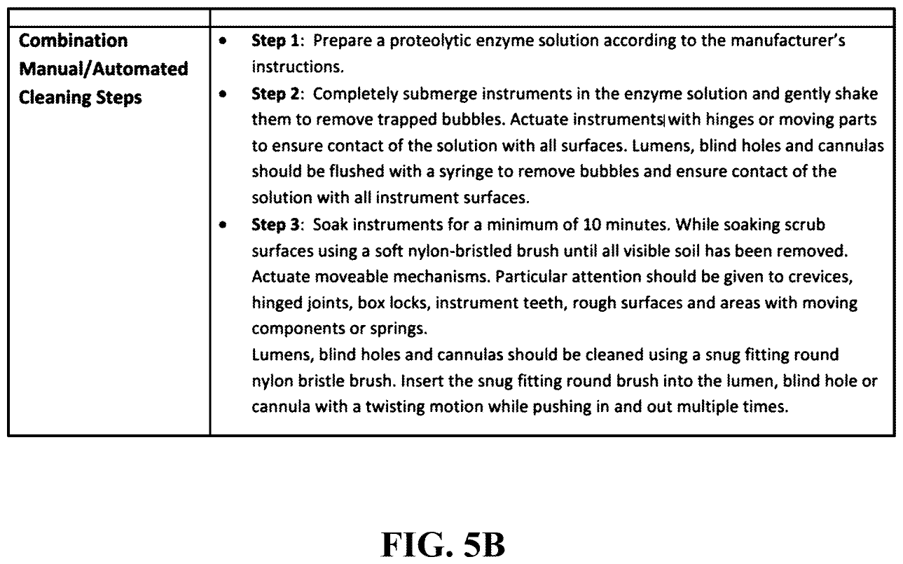

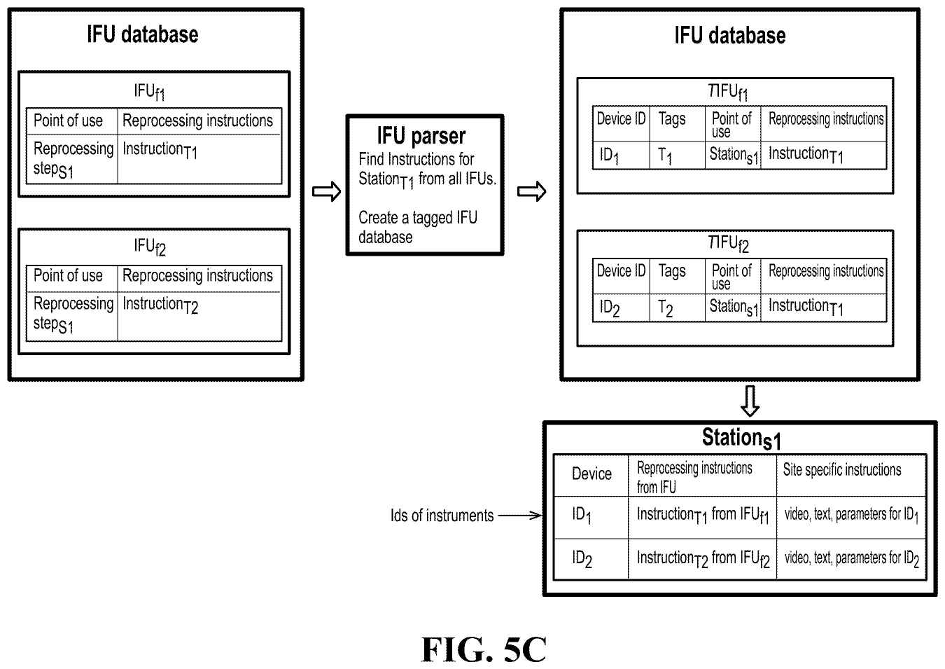

Context sensitive instructions. As indicated above, the sheer volume of instruments going through a SPD is quite large. Each instrument can have IFU that outlines procedures for cleaning and maintenance of that instrument and often the procedures and the settings for the various systems that are used within the workflow such as the sterilizer, are unique. As an example, in the decontamination area, each instrument should be washed and cleaned as per the IFU. But if the technician receives 150 instruments from a case and if he or she needs to turn that around in 30 mins, mistakes and errors can occur. The quality and speed of cleaning depends on the expertise, knowledge and training of the technician or the operator. Thus, it is advantageous to provide just the right information at the right location. FIG. 5A outlines such a concept where relevant information becomes available at the appropriate stations. In an initial step, within the SSR, and IFU database is created by downloading the manuals of each instrument. Each IFU is parsed and the parsed information is tagged according to the stations and procedures in that particular facility. This can result in sections for example tagged such that the instructions are appropriate for the washing station or for the sterilization station.

There are many software programs or modules that can be used to parse and tag the IFUs. Typically, document parsing is implemented by defining a set of parser rules. These predefined parser rules can be made available to the site where the SSR is installed as one of the software utilities. This software utility can allow customization of the parser rules such that the result is more relevant to the site that is deploying the SSR. Creation of parser rules as mentioned earlier, can be done apriori. Several tools, such as the commercially available “Docparser” can be used to create the parsing rules. The IFUs fortunately tend to fall in one of these categories of being structured or quasi-structured. The nature of the IFUs also allows the use of machine learning and artificial intelligence to create the parser rules. Thus, with the software utility, parsing rules can be set up to associate instructions in the IFU to a specific station such as the washing or the sterilization station. From time to time, the parser rules can have to be updated as new instruments and new terms are introduced. In such cases, the manufacturer can send these terms to the organization responsible for the parser rules which can subsequently update the rules and send out updates to the various sites using the SSR system. FIG. 5B illustrates these concepts in more detail. This figure is a screenshot from an IFU of an instrument. It demonstrates a typical style used for IFUs. In the left column, the reprocessing step is identified. The right column contains some of the instructions that need to be followed during the cleaning of decontamination step. It can be seen here that the instructions are quite prescriptive. In fact, the draft guidance from the FDA “processing/Reprocessing Medical Devices in Health Care Settings: Validation Methods and Labeling” recommends that a specific document called the “Technical Information Report” published by the Association of Medical Instruments (AAMI), be followed while developing labeling instructions for reusable medical devices. Thus, parsing software can be written to find information specific to a station. In the parlance of the FDA, these stations are called “Point of use”.

FIG. 5C provides a more detailed view of an implementation. The IFU databases for each instrument that a reprocessing facility acquires, can be downloaded and stored in the internal storage of the SSR system. The figure shows two IFUs IFU_{f1} and IFU_{f2}, each with a set of instructions for each step in the reprocessing workflow. A parser tool can be utilized to search for specific words and phrases within each IFU. An example of a parser tool is the search and find tools within a PDF reader. This tool can be implemented within the computing engine of the SSR. Subsequently, a tagged IFU database is created by a software program that includes the information obtained by the parser. This software program can associate tags and device identifiers to the information obtained from the parser. The tags can provide information that is specific to the facility. Additionally, a unique device identifier (UID) tag can also be associated with the tags and the information obtained from the parser. With this and other information a tagged IFU database can be create as illustrated in FIG. 5C. Thus, referring to the figure, TIFU_{f1} is an example of a tagged IFU database that has parsed instruction T1 from IFU_{f1} for a reprocessing step s1. Tag T₁ and identifier ID₁ has been associated with this instruction. Once this tagged IFU database is created and stored within the internal storage of the SSR, when instruments present themselves at a station, an initial step is to recognize the instruments. This can be accomplished on a subset by subset basis as markings on each subset can be used to retrieve that information. This assumes that all instruments in a subset that was used were returned and kept together as a set. In today's environment, the operator or technician in the decontamination station can have to sort the instruments back into their subsets. In more advanced methods as discussed below, each instrument can have a passive identifier such as an RFID chip that would allow sensors to recognize the instruments. Once recognized, the SSR can aggregate the instructions from one or multiple IFUs as the device identifier can already be associated with the instructions previously and display in on a screen associated with a specific station.

In a variation of this concept, the SSR can allow inclusion of site-specific testing, maintenance and other instructions along with the instructions provided by the manufacturer, generated by the technicians or the operators. Often the technicians or operators discover techniques or add detail that are not described in the IFU. For example, each instrument can be required to interface with the testing and wash stations in specific ways, and the database can be designed to hold the information about the pose angle and orientation of tools required for each step. As a further example, the system can contain measurement parameters (previously stored as a result of prior testing and verification runs) for each test such as integration time, illumination brightness, optical wavelength, etc. Additional information in various formats such as video clips, images, computer instructions or other forms of input can be added to a particular tagged segment of the IFU. Thus, as illustrated in FIG. 5C, at each station, the instructions as written in the IFU can be displayed along with site specific instructions added previously. A good example can be for an intricate instrument having lumens. The IFU can specify how to take apart and assemble the lumen however modified techniques discovered by the technicians can be more descriptive. To allow input of site-specific instructions, the information and instruction station can be equipped with accessories such as a camera that can be used to capture clips or images. This data can be stored along with the manufacturer's instructions and can be displayed the next time the specific instrument is handled at that particular station. In addition, these site-specific instructions can be provided to the manufacturer as feedback that can help in improving the IFU. The additional information can be used in various ways including but not limited to providing additional information to the operators and technicians, providing calibration information to the sensing and other types of equipment utilized within the reprocessing workflow and also providing computer instructions to automated equipment such as robotic systems, designed to automatically accomplish the reprocessing steps.

In another embodiment, the above capability described in FIG. 5C can regularly check for updates to the IFU. In many cases, IFUs can be updated by the manufacturer but the updates go unnoticed. In FIG. 5C, a software agent running within the SSR system can constantly check the revision of the IFU currently being used vs. the latest revision of the IFU published by the manufacturer. The software agent can initiate a download file action from the manufacturers web site and check the IFU revision number to ensure that the latest revision is being used. The parser under a different set of parser rules, can be used to compare the two versions of the IFUs and display the differences on a station when an instrument is recognized at a station.

Thus, in summary this section describes a method to parse manufacturer's instructions, add site specific information, associate that information to a specific instrument and display relevant information according to the station or the step in the workflow.

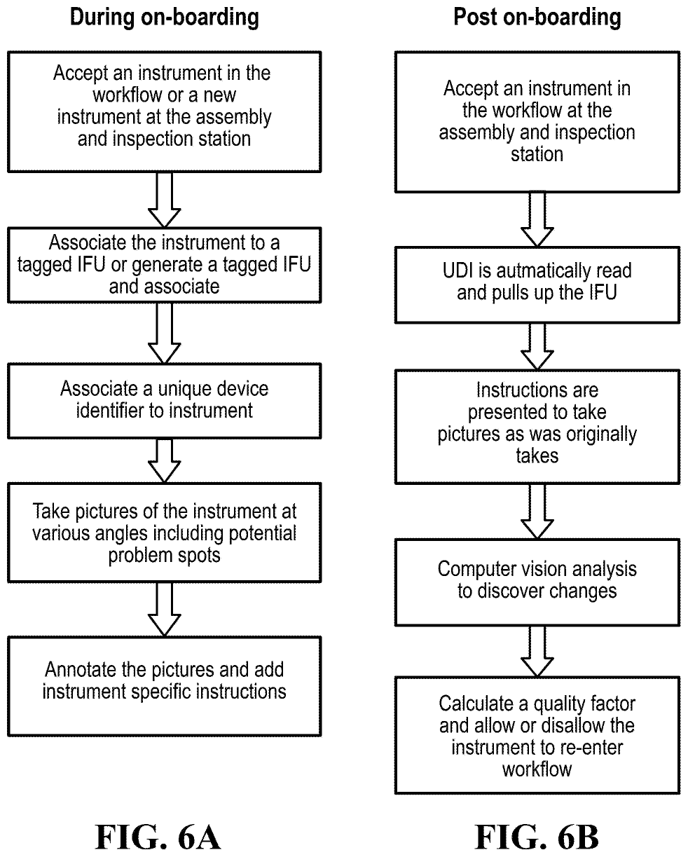

Onboarding system and building an interactive database. Many efficiencies can be realized by building a site-specific inventory that also can include a visual history of each instrument. Site specific inventories do exist today however they are typically not interactive and/or require manual oversight. In addition, it is also not typical to associate the inventories to the IFUs or to the current state of the instrument. The concept outlined in FIGS. 6A and 6B allows the creation and use of an interactive inventory database.

FIG. 6A outlines how to onboard the instruments and create an interactive inventory database. Although the onboarding process can occur in various stations (including at its own dedicated station), one convenient location is at the assembly and inspection station. Typically, at the assembly and inspection location, each instrument is handled by a human operator in today's environment. In an initial step, an instrument is accepted at this station either while the instrument is within a workflow cycle or while it can be getting inserted into the workflow cycle if new. In a subsequent step, the IFU for that instrument is downloaded and a tagged IFU is generated as in the previous discussion. The tagged IFU is associated with a specific instrument with a unique device identifier (UDI). In some cases, the instrument can already have an UDI. If not, an UDI number can be coupled to the instrument in various ways including laser etching. In some cases, a passive writable microchip which as a miniature RFID chip, can be coupled to the instrument by embedding the chip within the body of the instrument with epoxy. Once the UDI is associated with the instrument, a visual documentation of the instrument can be generated by taking photographs under known conditions such as lighting conditions, position of the instrument etc. The human operator's skill and knowledge can be utilized to position the instrument in specific positions that make the instrument identifiable by a computer vision system or instructions for how to present the instrument to the sensor can be conveyed to the user by querying the database. Additionally, the operator's skill and historical data can be also utilized to take photographs of “problem spots” such as hinges or edges or more generally, known points of failure. These images and the historical data can be stored in the interactive database. Subsequently, the pictures can be annotated or specific instructions for cleaning and maintenance can be included such that the next time this instrument comes through the workflow, the inspections can occur in a similar manner and a visual reference is available to compare against. The instructions can be stored in the inventory database or as described earlier, can be displayed in the tagged IFU database. Regardless of where the information is stored, the SSR can collect the relevant information from the various databases and present it to the operator when needed.

FIG. 6B outlines a candidate workflow after the onboarding has occurred. Here an instrument with a UDI is accepted at the assembly and inspection station. The instruction and information session at the station can be coupled to a UDI reader. Once the UDI is read, the tagged IFU and the information from the interactive database is accessed and information is displayed on the screen. Specific to the assembly and inspection station, the information can include specific instructions for the care and maintenance of the instrument. The instructions can also guide the operator to take pictures in the same conditions as was used during the onboarding stage. Once the pictures are taken, computer vision analysis can be used to analyze differences from prior images. A multitude of computer vision and sensing algorithms can be employed at this stage for change detection or identification purposes. Binary matching algorithms such as BRIEF and SURF can be used to generate codes to rapidly match instrument shape against reference data to confirm the identity of the instrument. Appearance of rust can be revealed through colorimetric analysis and used to detect problems with water quality. Pitting and cracking can be revealed from polarized light scattering data. The SSR can be programmed to generate an alert and highlight and log the location where changes in the instrument fail a present test criteria. The technician or an operator can now have to make a decision whether to allow the instrument to be used or pull the instrument out of the workflow.

To automate or augment the decision making process, a quality factor can be calculated for each instrument; the quality factor can reflect the state of the instrument. The quality factor can be defined differently for each instrument and can also become sophisticated over time with the availability of more that can include feedback from the surgical staff. The quality factor can be a single scalar value or a list of values that capture quality factors across many dimensions. As one example, a scissor can start out with a quality factor of 100 each for sharpness and pitting when it is new in an arbitrary scale. Assuming that the manufacturer recommends sharpening every 20 uses, the quality factor can be reduced by 5 points every use. If a threshold of 60 is used as a trigger level to alert the operator, then in 12 uses, an automatic warning can be initiated by the SSR. In a more advanced quality factor calculation for this scissor, the computer vision analysis can be programmed to identify rust or pits in the working part of the instrument. The manufacturer can specify that each area with rust or pits in the working area, decreases the quality factor by some number for example 10. A threshold of 80 can be set for pitting as a level that triggers an alert. Thus, two areas could bring the quality factor down and trigger the alert. In an even more sophisticated calculation, the size of the rust spot or the pit can also decrease the quality factor. The location of the rust or pit can also be considered. Thus, over time with various pieces of data including the feedback from the surgical team, the expert knowledge of the operator and the knowledge of the manufacturer, a robust quality factor can be defined and propagated to all facilities using the SSR.

Thus, the onboarding process and the interactive database provides a technique to build a database with information to improve the quality and efficiency of the reprocessing workflow. As will be described further below, the SSR system architecture provides a way to aggregate information about a specific type of instrument from one or multiple sites that use the SSR system. Thus, robust data about various parameters such as the wear and tear of the instrument, the points of failure, the effectiveness of the maintenance routine, can be gathered and used in various ways. This data can be used locally within a site for improving the efficiency and quality of the local procedures or it can be used in a global fashion as a feedback to the manufactures. As a result, many advantages can be realized such as improved maintenance instructions, improved inspection techniques, improved IFUs or potentially improved designs of the instruments. When such global information is realized, the information can be propagated to each of the sites for adoption.

Enhanced inspection and maintenance system. The concepts described above lend itself to an enhanced inspection and maintenance system. Elements of this system were described earlier. One aspect of this system is the collection and aggregation of data from multiple sources. Some sources of data and the type of data include maintenance and quality records from each institution, video history and image based analysis of instruments from the institutions, information including device malfunctions and adverse events caused or suspected due to a device from the manufacturer and user facility device experience (MAUDE) database, data from the global unique device identification database (GUDID) which included a unique device identifier for each device. As will be described below and as is illustrated in FIG. 8A, this information from these various sources can be collected by the external central server.

A second aspect of this system is the analysis of the data. Various types of analysis can be implemented. The maintenance and quality records for each type of instrument from the various institutions can be aggregated and one or multiple conclusions can result from this aggregation and analysis. For example, for a particular type of scissors, the analysis can reveal when the hinge joints begin to give out or when the sharpness of a particular type of scalpel begins to dull. The analysis of the video images of a particular type of instrument from various institutions can reveal information such as typically where biofilm occurs or where rust begins to form. The information from the MAUDE database can reveal a failure mode that may not have been anticipated. An example of such a failure mode can be that the lenses of an endoscope cracked during a surgery. These analyses can be supported by various software tools such that the analyses are done automatically once initiated. Various types of software tools can be written including those that use machine learning and AI to conduct the analysis. ML and AI techniques are well suited for this type of analysis as in general, the analysis can consist of spotting trends or spotting dependence of on a certain set of reprocessing parameters. Ample annotated training data can be made available to the ML and AI algorithms as the technicians and operators at various institutions can be recruited to gather such data as part of the interaction with the SSR system.

A third aspect is the generation of instructions. The analysis of information can lead to specific recommendations and instructions. As an example, an IFU for a particular instrument can suggest a few different alternative types of disinfectant solution however through data aggregation and analysis, it can be discovered that a particular type is more effective. This can then lead to generating a recommendation to use that type of disinfectant. Thus, analysis can result in a recommendation that can be pushed out to the various institutions. The recommendation or new instruction can appear as another field in the display of instructions (FIG. 5C).

A fourth aspect is the verification of performance of IFU steps. Within the scope of verification, one of the initial steps is to define the requirements for handling each instrument at each reprocessing step. Typically, requirements are developed by taking into account various inputs such as IFUs, historical failure modes etc. Typically also, corner cases are not accounted because during the requirements development process, full knowledge of how an instrument is used and reprocessed and the conditions under which it is used or reprocessed is not known. With the SSR system, multiple sources of data become available to overcome some of these shortcomings. As an example of an implementation to identify failure modes, for a particular tool used across various institutions, analysis (discussed in the first aspect above) of the test records from each institution, data from the MAUDE and other databases, can be aggregated for that specific tool. If a failure mode is identified, a new requirement for a reprocessing step can be revealed. The central computing engine can highlight such a failure following which a requirement for testing can be created either by a human operator or by a software agent capable of taking in failure data and creating a requirement. The failure modes can also be reported to the manufacturer which can result in the manufacturer updating the IFU. A typical next step in the verification process is to develop protocols to be followed for each instrument at each reprocessing workflow step. An example protocol for a pair of scissors at the inspection station, can be to open the pair as wide as possible and visually inspect the area near the hinge. These protocols are generally developed individually by each institution. With the SSR system and the architecture illustrated in FIG. 7 and FIG. 8A, at least two advanced concepts become possible. The first is that the protocols can be shared between institutions, thus laying the framework for standardized testing. Second, with the ability to collect data about the success and failure of the executing these protocols, the protocol that has the best success rate can be propagated. Advantageously, each station can also be outfitted with appropriate sensors such that a documentation of the execution of the steps in the protocol can be generated. An example is now provided to illustrate this aspect. For an instrument with lumens such as an endoscope, it is important that the lumens be disassembled (in case there are multiple lumens) and the inside of the lumens be cleaned with special brushes. In this case, a camera installed over the washing station can be used to obtain images of this step being performed in the following manner. When the instrument arrives at the decontamination station, the instrument can be recognized as discussed previously. The IFU and other site-specific information is automatically recalled on the screen. Site-specific information, as described previously, can include images, video clips taken previously to explain instructions in more detail. In the case of the instrument with lumens, the SSR system can then prompt the operator or technician to disassemble the lumens and position the lumens so that the camera can acquire an image of the two lumens. The same or a different camera can also be positioned to capture the action of scrubbing the inside of the lumens with a special brush in a video clip. A software agent can be running in the background to accept these images and perform image or video-based analysis and to make a determination if the steps of removing the two lumens and brushing the inside of the lumens was in fact carried out. The image or video-based analysis can range from a simple analysis to more complex. An example of a simple analysis can be that the analysis simply confirms that it can discern two long and separate cylindrical objects (the two lumens) and a third object that looks like a brush in the vicinity of each other in the same image frame. It can be more complex in that the brush can be coupled with a reflector and the camera system and associated software can be able to track the position of the reflector in relation to a lumen. Detection of a rhythmic motion of the brush can then be utilized as the signal that the inside of the lumen was cleaned. Thus, each station can be equipped with appropriate sensors such that confirmation of the completion of the protocols at one or multiple stations can be obtained. The records of performance can also be archived for analysis at a later time. Another example of how an inspection step is recorded is now provided. In the reprocessing workflow, peel packs sent to the surgical suite must be inspected to ensure that the peel pack is not torn or damaged; typically this is done through a visual inspection and no record of performing this step is kept. To improve this situation, a camera can be provided at the back table (assuming that the back table is in the sterile field), and the scrub nurse can place the peel pack in the imaging field of the camera and obtain images of the peel pack from different angles. These images can be stored as a matter of record along with the visual inspection. However, an image analysis routine can also be initiated whenever such images are obtained where the routine can analyze the image for tears in the peel pack.

Surgical supply and reprocessing system (SSR). FIG. 7 provides a block diagram of the surgical supply & reprocessing system (SSR). This system can consist of generic computer with sufficient computing, storage and networking capabilities to support several modules and functions. The computation engine can support computer vision analysis, quality factor computations, probability of usage calculations, dynamic packaging instructions and a search engine to search with the tagged IFU database. Other computations can also be supported. The SSR system can also include memory that stores the tagged IFU database, the interactive database and other data. A graphics engine can also be included to support provide visual instructions or renderings that can be used to provide instructions at any of the stations as required.

The SSR system supports input and output to one or multiple sources. It can exchange data with the EMR system for example through a common programming interface. The SSR system can be coupled to one or multiple instruction and information stations. These stations can include a computer with necessary peripherals. These stations can also include sensors such as the UDI sensor. The stations can also be customized to do specific tasks such as sensing for biomass after washing. The SSR system can also be coupled to an onboarding system as discussed above. The SSR system can also be connected via the cloud to a central computing facility enabling data and analysis from separate institutions to be shared. Bi-directional communication between the cloud and the station is used control test parameters unique to each instrument and signal when measurements are completed on each UDI-labeled device. Workflows are customized based on customer input and from past data collected from the instruments. Representing each instrument as a vector simplifies the computation of processing cost at all workflow steps to a matrix multiplication. The connection to the cloud system can also enable sharing or more efficient use of resources such as when instrument trays are supplied by external suppliers.

Other than planning and monitoring the reprocessing SSR system can also generate several types of outputs such as compliance reports, inventory assessment etc. Data such as the history of the quality factor and associated images taken during every pass of the instrument through the assembly and inspection station can be included in such reports.

While FIG. 7 provides a block diagram of the SSR system as it can be configured within a site, FIG. 8A provides a broader view of the architecture. Here, an external central server can be networked to one or several SSR systems placed in various sites, Site A to Site N. The flow of information is bidirectional between the on premise SSRs and the external central server. Examples of interactions between the SSRs and the external central server have been discussed above. To perform the various functions some of which have been mentioned above, the external central server can include within its hardware configuration a central memory and a central computing engine. Thus, the analysis of data arising from multiple sites, can be implemented within this computing engine. The external server can (as the individual SSR can as well), interact and accept data from one or several databases such as the MAUDE database. This provides the ability to monitor information such as recall information, failure modes etc. The external server can also interact with one or multiple manufacturers accepting data such as updated IFUs and providing data such as recommendations to improve the IFUs.

The networking architecture enables another concept illustrated in FIG. 8A and FIG. 8B. As seen in FIG. 8A, the external central server can interact with an off-premise reprocessing and storage facility that includes an SSR system located in such a site. The difference between the on-premise and off-premise system, is that the on-premise systems are located in sites that also includes the surgical suite. In contrast, the off-premise site can include a storage facility and/or a reprocessing facility as well. This type of architecture allows several advantages to be realized. One advantage is that each site such as the hospital or outpatient clinic (one of the sites A through N), can reduce the allotment of resources such as labor, lab space or storage space to the reprocessing function. Another advantage is that resource balancing of the instruments can be more efficiently done across the various sites. Yet another advantage is that the testing and reprocessing can become more standardized.

FIG. 8B illustrates how the architecture of FIG. 8A can be used to further optimize the reprocessing workflows. Here some of the sites A through N can retain the ability to perform the reprocessing function. This the external central server can input the surgery schedule and the subset configurations for the next D days from the N sites. Subsequently, a master inventory database can be created or updated containing information about where each instrument is and what state of use or reprocessing it is in. Based on the schedule, the subset configuration, and the master inventory, a optimization routine can be implemented to determine where best to reprocess the instruments on-premises in one of the sites A through N or in the off-premise site to achieve a result such as minimal cost of reprocessing and storage. As an example, instruments that are needed within the next 24 hours can be processed in one of the on-premise sites however instruments that are needed beyond the 24 hour window can be processed on the off-premise facility.

What is claimed is:

1. A surgical supply and reprocessing method comprising:
accepting data about a procedure, patient, the surgical team or other relevant information from at least one source;
calculating the probability that a surgical instrument will be used;
optimizing the surgical instrument configuration based on an algorithm;
calculating the cost of the configuration
choosing a minimal cost option;
displaying a candidate configuration to a qualified person;
personalizing the option presented;
developing package instructions;
inspecting and packaging the instruction
sterilizing the surgical instruments
familiarizing operating room staff with the instruments; and
transporting the surgical instruments post operation.
